(19) United States
(12) Patent Application Publication
(10) Pub. No.: US 2010/0092401 A1
(43) Pub. Date: Apr. 15, 2010

(54) IMMUNE RESPONSE MODIFIER FORMULATIONS

(75) Inventors: Janet A. Vallejo, Eagan, MN (US); Suzanne S. Leung, Woodbury, MN (US); Patrick J. Roddy, Mahtomedi, MN (US); Terri F. Busch, St. Paul, MN (US)

Correspondence Address: EDWARDS ANGELL PALMER & DODGE LLP, P.O. BOX 55874, BOSTON, MA, 02205, US

(73) Assignee: Graceway Pharmaceuticals, LLC, Bristol, TN (US)

(21) Appl. No.: 12/309,367

(22) PCT Filed: Jul. 12, 2007
(86) PCT No.: PCT/US2007/016029

§ 371 Date: Dec. 15, 2009

Related U.S. Application Data
(60) Provisional application No. 60/807,669, filed on Jul. 18, 2006.

(51) Int. Cl. A61K 9/12; A61K 31/437; A61P 37/02

(52) U.S. Cl. 424/45; 514/293

(57) ABSTRACT

The present invention provides a pharmaceutical foam formulation. Generally, the formulation includes a therapeutically effective amount of imiquimod and a fatty acid.

### FIELD OF INVENTION

The present invention provides a pharmaceutical foam formulation. Generally, formulation includes a therapeutically effective amount of imiquimod and fatty acid.

### BACKGROUND

There has been a major effort in recent years to discover new drug compounds that act by stimulating certain key aspects of the immune system, as well as by suppressing certain other aspects (see, e.g., U.S. Pat. Nos. 6,039,969 and 6,200,592). These compounds, sometimes referred to as immune response modifiers (IRMs), appear to act through basic immune system mechanisms known as toll-like receptors to induce selected cytokine biosynthesis and may be used to treat a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis), and TH₂-mediated diseases (e.g., asthma, allergic rhinitis, atopic dermatitis), and are also useful as vaccine adjuvants. Unlike many conventional anti-viral or anti-tumor compounds, the primary mechanism of action for IRMs is indirect, by stimulating the immune system to recognize and take appropriate action against a pathogen.

Many of the IRM compounds are imidazoquinoline amine derivatives (see, e.g., U.S. Pat. No. 4,689,338), but a number of other compound classes are now known as well (see, e.g., U.S. Pat. Nos. 5,446,153; 6,194,425; and 6,110,929).

Pharmaceutical compositions containing IRM compounds are disclosed in U.S. Pat. Nos. 5,238,944; 5,939,090; and 6,425,776; European Patent 0 394 026; and U.S. Patent Publication 2003/0199538. The IRM compound, 1-(2-methylpropyl)-1H-imidazo[4,5-c]quinolin-4-amine, also known as imiquimod, has been commercialized in a topical formulation, ALDARA, for the treatment of actinic keratosis, basal cell carcinoma, or anogenital warts associated with human papillomavirus.

However, providing therapeutic benefit by topical application of an IRM compound for treatment of a particular condition at a particular location or of a particular tissue can be hindered by a variety of factors, such as, for example, chemical degradation of the IRM compound and/or other ingredients, and physical instability of the composition (e.g., separation of components, thickening, precipitation or agglomerization of active ingredient, and the like).

Therefore, there is a continuing need for new and/or improved IRM formulations.

### SUMMARY

It has been found that imiquimod can be formulated as a pharmaceutical foam formulation.

Accordingly, the present invention provides pharmaceutical foam formulation. Generally, the formulation includes a therapeutically effective amount of imiquimod and a fatty acid.

In another aspect, the present invention also provides a method of treating actinic keratosis. Generally, the method includes applying a formulation that includes a therapeutically effective amount of imiquimod and a fatty acid to the skin of a subject in need of such treatment.

In another aspect, the present invention also provides a method of treating basal cell carcinoma. Generally, the method includes applying a formulation that includes a therapeutically effective amount of imiquimod and a fatty acid to the skin of a subject in need of such treatment.

In another aspect, the present invention also provides a method of treating anogenital warts. Generally, the method includes applying a formulation that includes a therapeutically effective amount of imiquimod and a fatty acid to the skin of a subject in need of such treatment.

In another aspect, the present invention also provides a method of inducing interferon biosynthesis. Generally, the method includes applying a formulation that includes a therapeutically effective amount of imiquimod and a fatty acid to the skin of a subject in need of interferon biosynthesis.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

It has been found that imiquimod may be formulated in an emulsion-based foam. Generally, the foam formulation includes a therapeutically effective amount of imiquimod and a fatty acid.

As used herein, “a,” “an,” “the,” “at least one,” and “one or more” are used interchangeably. Thus, for example, a formulation comprising “a” fatty acid can be interpreted to mean that the formulation includes at least one fatty acid.

Also herein, the recitations of numerical ranges by endpoints include all numbers subsumed within that range (e.g., 1 to 5 includes 1, 1.5, 2, 2.75, 3, 3.80, 4, 5, etc.).

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic and scalemic mixtures of the enantiomers.

The formulation includes the imidazoquinoline amine 1-(2-methylpropyl)-1H-imidazo[4,5-c]quinolin-4-amine, also referred to as imiquimod, the synthesis of which is described at, for example, U.S. Pat. No. 4,689,338, Example 99.

The amount of imiquimod present in a composition of the invention will be an amount effective to treat, prevent the recurrence of, or promote immunity to the targeted disease state, or to improve skin quality. In certain embodiments, the total amount of imiquimod can be at least 0.5 percent by weight, and no more than 9 percent by weight, based on the total weight of the composition (unless otherwise indicated, all percentages provided herein are weight/weight with respect to the total weight of the composition), although in some embodiments the composition may contain an amount of imiquimod outside of this range. In some embodiments, the composition includes imiquimod in an amount of from about 1.0% to about 6.0%. For example, the composition may include imiquimod at a concentration of 1%, 3%, 5%, or 6%.

The foam compositions of the invention may include one or more additional excipients such as, for example, a fatty acid, a preservative, a thickener, an emulsifier, a solubilizing agent, an emollient, or a humectant.

The formulations of the invention include one or more fatty acids. As used herein, the term “fatty acid” means a carboxylic acid, either saturated or unsaturated having 6 to 28 carbon atoms, such as, for example, from 10 to 22 carbon atoms. Fatty acids suitable for use in the formulations described herein include those that can aid in solubilizing imiquimod. Suitable fatty acids include, for example, isostearic acid, oleic acid, myristic acid, palmitic acid, palmitoleic acid, margaric acid, stearic acid, linoleic acid, linolenic acid, or mixtures thereof. In some embodiments, the formulation includes, for example, isostearic acid, oleic acid, or a mixture thereof. In one particular embodiment, the formulation includes isostearic acid.

The fatty acid is present in the formulation in an amount sufficient to solubilize imiquimod. In some embodiments, the total amount of fatty acid, is at least 0.05% by weight, at least 1.0% by weight, at least 3.0% by weight, at least 5.0%, at least 10%, at least 15%, or at least 25%, based on the total weight of the formulation. In certain embodiments, the total amount of fatty acid is at most 40% by weight, at most 30% by weight, at most 15% by weight, or at most 10% by weight, based on the total weight of the formulation.

For certain embodiments of the invention, the formulation further includes a propellant. In certain embodiments, the propellant is a hydrocarbon propellant. Suitable hydrocarbon propellants include, for example, lower alkanes, such as for example, propane and butane. Any combination of suitable propellants can be included in the formulation. For example, the formulation can include a combination of propane and butane. One embodiment includes a 50:50 combination of propane and butane. Another embodiment includes a 10:90 combination of propane and butane.

The total amount of propellant can be from about 2% to about 25%, although in some embodiments, the formulation can include a total amount of propellant outside of this range. In one embodiment, the total amount of propellant is about 5%. In another embodiment, the total amount of propellant is about 10%. In still another embodiment, the total amount of propellant is about 15%.

For certain embodiments of the invention, the formulation further includes a preservative system. The preservative system includes one or more compounds that inhibit microbial growth (e.g., fungal and bacterial growth) within the formulation (for example, during manufacturing and use). The preservative system will generally include at least one preservative compound, such as, for example, methylparaben, ethylparaben, propylparaben, butylparaben, benzyl alcohol, phenoxyethanol, and sorbic acid or derivatives of sorbic acid such as esters and salts. Various combinations of these compounds can be included in the preservative system. In some embodiments of the invention, the preservative system includes methylparaben, propylparaben and benzyl alcohol. In other embodiments of the invention, the preservative system includes methylparaben and benzyl alcohol.

In some embodiments of the invention, the preservative system is present in an amount of at least 0.01% by weight, such as for example, at least 0.02%, at least 0.03%, at least 0.04%, and at least 0.05%, by weight based on the total weight of the formulation. In other embodiments of the invention the preservative system is present in an amount of at most 3%, such as for example, at most 2.5%, at most 2.0%, at most 1.0%, at most 0.5%, at most 0.4%, at most 0.3%, and at most 0.2%, by weight based on the total weight of the formulation.

For certain embodiments of the invention, the formulation further includes an emulsifier. Suitable emulsifiers include non-ionic surfactants such as, for example, polysorbate 60, sorbitan monostearate, polyglyceryl-4 oleate, polyoxyethylene(4) lauryl ether, poloxamers, and sorbitan trioleate. In certain embodiments, the emulsifier is chosen from polysorbate 60, sorbitan monostearate, and mixtures thereof.

If included, the emulsifier is generally present in an amount of 0.1% to 10% by weight of total formulation weight, for example, from 0.5% to 5.0% by weight, and from 0.75% to 4.0% by weight. In certain embodiments, the amount of the emulsifier, if used, is present in an amount of at least 0.1% by weight, at least 0.5% by weight, at least 0.75% by weight, at least 1.0% by weight, at least 2.5% by weight, at least 3.5% by weight, at least 4.0% by weight, or at least 5.0% by weight, based on the total weight of the formulation. In certain embodiments, the amount of the emulsifier, if used, is present in an amount of at most 10% by weight, at most 5.0% by weight, or at most 3.5% by weight, based on the total weight of the formulation.

For certain embodiments of the invention, the formulation further includes a viscosity-enhancing agent. Examples of suitable viscosity enhancing agents include long chain alcohols, for example, cetyl alcohol, stearyl alcohol, cetearyl alcohol; cellulose ethers such as hydroxypropylmethylcellulose, hydroxyethylcellulose, hydroxypropylcellulose, and carboxymethylcellulose; polysaccharide gums such as xanthan gum; and homopolymers and copolymers of acrylic acid crosslinked with allyl sucrose or allyl pentaerythriol such as those polymers designated as carbomers in the United States Pharmacopoeia. In certain embodiments, the viscosity enhancing agent is xanthan gum.

In certain embodiments, the amount of the viscosity enhancing agent, when used, is at least 0.1% by weight, at least 0.2% by weight, at least 0.5% by weight, at least 0.6% by weight, at least 0.7% by weight, at least 0.9% by weight, or at least 1.0% by weight, based on the total weight of the formulation. In certain embodiments, the amount of the viscosity-enhancing agent, when used, is at most 10% by weight, at most 5.0% by weight, at most 3.0% by weight, at most 2.0% by weight, or at most 1.5% by weight, based on the total weight of the formulation.

For certain embodiments of the invention, the formulation further includes at least one emollient. Examples of suitable emollients include, but are not limited to, long chain alcohols, for example, cetyl alcohol, stearyl alcohol, cetearyl alcohol; fatty acid esters, for example, isopropyl myristate, isopropyl palmitate, diisopropyl dimer dilinoleate; medium-chain (e.g., 8 to 14 carbon atoms) triglycerides, for example, caprylic/capric triglyceride; cetyl esters; hydrocarbons of 8 or more carbon atoms, for example, light mineral oil, white petrolatum; and waxes, for example, beeswax. Various combinations of such emollients can be used if desired. In certain embodiments, the emollient is chosen from cetyl alcohol, stearyl alcohol, petrolatum, and mixtures thereof.

In certain embodiments, the amount of the emollient is at least 1.0% by weight, at least 3.0% by weight, at least 5.0% by weight, or at least 10% by weight, based on the total weight of the formulation. In certain embodiments, the amount of emollient is at most 30% by weight, at most 15% by weight, or at most 10% by weight, based on the total weight of the formulation.

In some embodiments, formulations of the invention are oil-in-water emulsions. The water used in these formulations is typically purified water

Optionally, a formulation of the invention can include additional pharmaceutically acceptable excipients such as humectants, such as for example, glycerin; chelating agents, such as for example, ethylenediaminetetraacetic acid; and pH adjusting agents, such as for example, potassium hydroxide or sodium hydroxide. In certain embodiments, the formulation includes glycerin.

In some instances, a single ingredient can perform more than one function in a formulation. For example, cetyl alcohol can serve as both an emollient and a viscosity enhancer.

Conditions that may be treated by administering imiquimod in a foam composition of the invention include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

(g) diseases associated with wound repair such as, for example, inhibition of keloid formation and other types of scarring (e.g., enhancing wound healing, including chronic wounds).

Additionally, the foam compositions may be useful for delivery of imiquimod as a topical vaccine adjuvant for use in conjunction with any material that raises either humoral and/or cell-mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

The methods of the present invention may be performed on any suitable subject. Suitable subjects include but are not limited to animals such as, for example, humans, non-human primates, poultry, fowl, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The formulations shown in Tables 1 and 2 below were prepared using the following general method.

Imiquimod/isostearic acid/benzyl alcohol premix preparation: Imiquimod was combined with isostearic acid and mixed with heating (about 55° C.) until the bulk of the imiquimod was dissolved. Benzyl alcohol was added and mixing was continued until all of the imiquimod was in solution.

Oil phase preparation: Cetyl alcohol, stearyl alcohol, white petrolatum, polysorbate 60, and sorbitan monostearate were added to the imiquimod/isostearic acid/benzyl alcohol premix and mixed with heating (about 55° C.) until all components were dissolved.

Aqueous phase preparation: Methylparaben and propylparaben were combined with water and a portion (about 50%) of the glycerin and mixed with heating (about 55° C.) until the parabens were dissolved. Separately, a dispersion of xanthan gum was prepared by combining xanthan gum with the remaining portion of glycerin and mixing until the xanthan gum was dispersed. The xanthan gum dispersion was slowly added with mixing to the parabens solution while maintaining the temperature at about 55° C. Mixing was continued until the xanthan gum was fully dispersed.

Phase combination: The aqueous phase was added to the oil phase at about 55° C. The mixture was homogenized for a minimum of 15 minutes. The resulting emulsion was cooled to ambient temperature and then placed in a glass jar. Table 1 below shows the composition (percentage weight-by-weight basis) of the formulations prior to the addition of the propellant.

Addition of propellant: Emulsion was placed in a plastic-coated glass vial. An aerosol valve, either a continuous valve or a metered-dose valve, was crimped onto the vial. The vial was charged with propellant using a pressure burette with a nitrogen headspace. The vial was then agitated to disperse the emulsion in the propellant. Table 2 below shows the composition (percentage weight-by-weight basis) of the formulations after addition of propellant.

TABLE 1
Formulations Prior to the Addition of Propellant

| Ingredient | No 1 | No 2 | No 3 | No 4 | No 5 | No 6 |
|---|---|---|---|---|---|---|
| Imiquimod | 5.88 | 5.88 | 5.56 | 5.26 | 5 | 5 |
| Isostearic acid | 29.41 | 29.41 | 27.78 | 26.32 | 25 | 25 |
| Benzyl alcohol | 2.35 | 2.35 | 2.22 | 2.11 | 2 | 2 |
| Polysorbate 60 | 4.00 | 4.00 | 3.78 | 3.58 | 3.4 | 3.4 |
| Sorbitan monostearate | 0.71 | 0.71 | 0.67 | 0.63 | 0.6 | 0.6 |
| Xanthan gum | 0.59 | 0.59 | 0.56 | 0.53 | 0.5 | 0.5 |
| Methylparaben | 0.24 | 0.24 | 0.22 | 0.21 | 0.2 | 0.2 |
| Propylparaben | 0.02 | 0.02 | 0.02 | 0.02 | 0.02 | 0.02 |
| Cetyl alcohol | 2.59 | 2.59 | 2.44 | 2.32 | 2.2 | 2.2 |
| Stearyl alcohol | 3.65 | 3.65 | 3.44 | 3.26 | 3.1 | 3.1 |
| Petrolatum | 3.53 | 3.53 | 3.33 | 3.16 | 3 | 3 |
| Glycerin | 2.35 | 2.35 | 2.22 | 2.11 | 2 | 2 |
| Water | 44.68 | 44.68 | 47.76 | 50.51 | 52.98 | 52.98 |

TABLE 2
Formulations After the Addition of Propellant

| Ingredient | No 1 | No 2 | No 3 | No 4 | No 5 | No 6 |
|---|---|---|---|---|---|---|
| Imiquimod | 5 | 5 | 5 | 5 | 4.25 | 4.75 |
| Isostearic acid | 25 | 25 | 25 | 25 | 21.25 | 23.75 |
| Benzyl alcohol | 2 | 2 | 2 | 2 | 1.7 | 1.9 |
| Polysorbate 60 | 3.4 | 3.4 | 3.4 | 3.4 | 2.89 | 3.23 |
| Sorbitan monostearate | 0.6 | 0.6 | 0.6 | 0.6 | 0.51 | 0.57 |
| Xanthan gum | 0.5 | 0.5 | 0.5 | 0.5 | 0.43 | 0.48 |
| Methylparaben | 0.2 | 0.2 | 0.2 | 0.2 | 0.17 | 0.19 |
| Propylparaben | 0.02 | 0.02 | 0.02 | 0.02 | 0.02 | 0.02 |
| Cetyl alcohol | 2.2 | 2.2 | 2.2 | 2.2 | 1.87 | 2.09 |
| Stearyl alcohol | 3.1 | 3.1 | 3.1 | 3.1 | 2.64 | 2.95 |
| Petrolatum | 3 | 3 | 3 | 3 | 2.55 | 2.85 |
| Glycerin | 2 | 2 | 2 | 2 | 1.7 | 1.9 |
| Water | 37.98 | 37.98 | 42.98 | 47.98 | 45.02 | 50.32 |
| 50:50 Propane:butane | 15 | 0 | 10 | 5 | 15 | 5 |
| 10:90 Propane:butane | 0 | 15 | 0 | 0 | 0 | 0 |

The formulations shown in Tables 3 and 4 below were prepared using the following general method.

Imiquimod/isostearic acid/benzyl alcohol premix preparation: Imiquimod was combined with isostearic acid and mixed with heating (about 55° C.) until the bulk of the imiquimod was dissolved. Benzyl alcohol was added and mixing was continued until all of the imiquimod was in solution.

Oil phase preparation: Polysorbate 60 and sorbitan monostearate were added to the imiquimod/isostearic acid/benzyl alcohol premix and mixed with heating (about 55° C.) until all components were dissolved.

Aqueous phase preparation: Methylparaben and propylparaben, if included, were combined with water and mixed with heating (about 55° C.) until the parabens were dissolved. Xanthan gum was added and the aqueous phase was mixed at about 55° C. until the xanthan gum was fully dispersed.

Phase combination: The aqueous phase was added to the oil phase at about 55° C. The mixture was homogenized for a minimum of 15 minutes. The resulting emulsion was cooled to ambient temperature and then placed in a glass jar. Table 3 below shows the composition (percentage weight-by-weight basis) of the formulations prior to the addition of the propellant.

Addition of propellant: Emulsion was placed in a plastic-coated glass vial. An aerosol valve, either a continuous valve or a metered-dose valve, was crimped onto the vial. The vial was charged with propellant using a pressure burette with a nitrogen headspace. The vial was then agitated to disperse the emulsion in the propellant. Table 4 below shows the composition (percentage weight-by-weight basis) of the formulations after addition of propellant.

TABLE 3
Formulations Prior to the Addition of Propellant

| Ingredient | No 7 | No 8 | No 9 | No 10 | No 11 | No 12 |
|---|---|---|---|---|---|---|
| Imiquimod | 5.88 | 5.88 | 5.56 | 5.26 | 5.26 | 1.05 |
| Isostearic acid | 29.41 | 29.41 | 27.78 | 26.32 | 26.32 | 5.26 |
| Benzyl alcohol | 2.35 | 2.35 | 2.22 | 2.11 | 2.11 | 0.42 |
| Polysorbate 60 | 4.00 | 4.00 | 3.78 | 3.58 | 3.58 | 0.72 |
| Sorbitan monostearate | 0.71 | 0.71 | 0.67 | 0.63 | 0.63 | 0.13 |
| Xanthan gum | 0.59 | 0.59 | 0.56 | 0.53 | 1.05 | 1.05 |
| Methylparaben | 0.24 | 0.24 | 0.22 | 0.21 | 0.11 | 0.11 |
| Propylparaben | 0.02 | 0.02 | 0.02 | 0.02 | 0 | 0 |
| Water | 56.80 | 56.80 | 59.20 | 61.35 | 60.95 | 91.26 |

TABLE 4
Formulations After the Addition of Propellant

| Ingredient | No 7 | No 8 | No 9 | No 10 | No 11 | No 12 |
|---|---|---|---|---|---|---|
| Imiquimod | 5 | 5 | 5 | 5 | 5 | 1 |
| Isostearic acid | 25 | 25 | 25 | 25 | 25 | 5 |
| Benzyl alcohol | 2 | 2 | 2 | 2 | 2 | 0.4 |
| Polysorbate 60 | 3.4 | 3.4 | 3.4 | 3.4 | 3.4 | 0.68 |
| Sorbitan monostearate | 0.6 | 0.6 | 0.6 | 0.6 | 0.6 | 0.12 |
| Xanthan gum | 0.5 | 0.5 | 0.5 | 0.5 | 1 | 1 |
| Methylparaben | 0.2 | 0.2 | 0.2 | 0.2 | 0.1 | 0.1 |
| Propylparaben | 0.02 | 0.02 | 0.02 | 0.02 | 0 | 0 |
| Water | 48.28 | 48.28 | 53.28 | 58.28 | 57.9 | 86.7 |
| 50:50 Propane:butane | 15 | 0 | 10 | 5 | 0 | 0 |
| 10:90 Propane:butane | 0 | 15 | 0 | 0 | 5 | 5 |

### Example 1

Male CD hairless rats (Charles River Laboratories, Wilmington, Mass.) weighing between 225-250 grams (approx. 8-10 weeks of age) are acclimated to neck collars for two consecutive days prior to dosing. On the day of the study, the rats are collared to prevent oral ingestion of the formulation, then treated topically with 45-47 mg of formulation from either Table 2 or Table 4 over an area of 6 approximately cm² at the right, lower back. The formulation is massaged into the skin using a nitrile-gloved finger for approximately 1-3 minutes. The rats are individually housed and remain collared for six hours.

After six hours, blood is collected by cardiac puncture under CO₂ anesthesia. The blood is allowed to clot at room temperature for approximately 20 minutes, then the serum is separated from the clot by centrifugation (Beckman Coulter Allegra 21R, S4180 horizontal rotor, Beckman Coulter, Inc. Fullerton, Calif.) at 2000 rpm for 10 minutes under refrigeration. The resulting serum is stored at −20° C. until analyzed for TNF-α and MCP-1 concentrations.

The animals are sacrificed and three 8 mm punch biopsies are obtained from each of two sites of each animal: the treatment site and opposite flank (left, lower back). The biopsy tissues are weighed and placed in a sealed 1.8 mL cryovial and flash frozen in liquid N₂. The frozen dermal tissue is suspended in 1.0 mL of RPMI medium (Protide Pharmaceuticals, St. Paul, Minn.) 10% fetal bovine serum, 2 mM L-glutamine, 1% penicillin/streptomycin, 5×10⁻⁵M 2-mercaptoethanlo and 1% protease inhibitor cocktail set III (Calbiochem/EMD Biosciences, San Diego, Calif.). The dermal tissue is homogenized on ice using a TISSUE TEAROR (Biospec Products, Inc. Bartlesville, Okla.) for approximately one minute. The dermal tissue supernatants are centrifuged (Beckman Coulter Allegra 21R, S4180 horizontal rotor) at 4800 rpm for 10 minutes under refrigeration to pellet cell debris. The supernatants are collected and stored at −20° C. until analyzed for TNF-α and MCP-1 concentrations.

TNF-α and MCP-1 concentrations are assayed by ELISA (TNF-α, BD Pharmingen, San Diego, Calif.; MCP-1, Biosource International, Camarillo, Calif.) according to manufacturer's specifications. ELISA plates are read on a SpectraMax microassay plate reader (Molecular Devices Corp., Sunnyvale, Calif.) and software SOFTMAX PRO is utilized for curve fitting of absorbance readings. Results for serum concentrations of TNF-α and MCP-1 are expressed in picograms/milliliter (pg/mL) for serum. Results for dermal tissue concentrations are expressed as pg per 200 mg of tissue.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A pharmaceutical foam formulation comprising a therapeutically effective amount of imiquimod and a fatty acid.

2. A formulation according to claim 1 wherein imiquimod is present in an amount of at least 1% by weight based on the total weight of the formulation.

3. A formulation according to claim 1 wherein imiquimod is present in an amount of at least 3% by weight based on the total weight of the formulation.

4. A formulation according to claim 1 wherein imiquimod is present in an amount of at least 5% by weight based on the total weight of the formulation.

5. A formulation according to claim 1 wherein imiquimod is present in an amount of at most 6% by weight based on the total weight of the formulation.

6. A formulation according to claim 1 wherein the fatty acid is present in an amount of at least 5% by weight based on the total weight of the formulation.

7. A formulation according to claim 1 wherein the fatty acid is present in an amount of at least 20% by weight based on the total weight of the formulation.

8. A formulation according to claim 1 or claim 7 wherein the fatty acid is present in an amount of at most 30% by weight based on the total weight of the formulation.

9. A formulation according to claim 1 wherein the fatty acid is selected from the group consisting of isostearic acid, oleic acid, and mixtures thereof.

10. A formulation according to claim 9 wherein the fatty acid is isostearic acid.

11. A formulation according to claim 1 further comprising a propellant.

12. A formulation according to claim 11 wherein the propellant is present in an amount of at least 5% by weight based on the total weight of the formulation.

13. A formulation according claim 11 wherein the propellant is present in an amount of at least 10% by weight based on the total weight of the formulation.

14. A formulation according claim 11 wherein the propellant is present in an amount of at least 15% by weight based on the total weight of the formulation.

15. A formulation according to claim 11 wherein the propellant is a hydrocarbon.

16. A formulation according to claim 15 wherein the hydrocarbon is selected from the group consisting of propane, butane, and mixtures thereof.

17. A formulation according to claim 1 further comprising water.

18. A formulation according to claim 1 further comprising a preservative system.

19. A formulation according to claim 18 wherein the preservative system is present in an amount of at least 0.1% by weight based on the total weight of the formulation.

20. A formulation according to claim 18 wherein the preservative system is present in an amount of at most 3% by weight based on the total weight of the formulation.

21. A formulation according to claim 18 wherein the preservative system comprises methylparaben, propylparaben, benzyl alcohol, or mixtures thereof.

22. A formulation according to claim 1 further comprising an emulsifier.

23. A formulation according to claim 22 wherein the emulsifier is present in an amount of at least 0.5% by weight based on the total weight of the formulation.

24. A formulation according to claim 22 wherein the emulsifier is present in an amount of at most 5% by weight based on the total weight of the formulation.

25. A formulation according to claim 22 wherein the emulsifier is selected from the group consisting of polysorbate 60, sorbitan monostearate, and mixtures thereof.

26. A formulation according to claim 1 further comprising a viscosity enhancing agent

27. A formulation according to claim 26 wherein the viscosity enhancing agent is present in an amount of at least 0.5% by weight based on the total weight of the formulation.

28. A formulation according to claim 26 wherein the viscosity enhancing agent is present in an amount of at least 1% by weight based on the total weight of the formulation.

29. A formulation according to claim 26 wherein the viscosity enhancing agent is present in an amount of at most 6% by weight based on the total weight of the formulation.

30. A formulation according to claim 26 wherein the viscosity enhancing agent is selected from the group consisting of cetyl alcohol, stearyl alcohol, xanthan gum, and mixtures thereof.

31. A formulation according to claim 1 further comprising an emollient.

32. A formulation according to claim 1 further comprising a humectant.

33. A formulation according to claim 17 wherein the formulation is an oil-in-water emulsion.

34. A packaged formulation comprising a formulation of claim 1 enclosed in an aerosol vial.

35. A packaged formulation according to claim 34 wherein the aerosol vial is equipped with a metering valve.

36. A method of treating actinic keratosis, the method comprising applying a formulation of claim 1 to the skin of a subject.

37. A method of treating basal cell carcinoma, the method comprising applying a formulation of claim 1 to the skin of a subject.

38. A method of treating anogenital warts, the method comprising applying a formulation of claim 1 to the skin or mucosal surface of a subject.

39. A method of inducing interferon biosynthesis, the method comprising applying a formulation of claim 1 to the skin or mucosal surface of a subject.
